(19) United States
(12) Patent Application Publication
(10) Pub. No.: US 2025/0259727 A1
(43) Pub. Date: Aug. 14, 2025

(54) MACHINE-LEARNING-BASED MEAL DETECTION AND SIZE ESTIMATION USING CONTINUOUS GLUCOSE MONITORING (CGM) AND INSULIN DATA

(71) Applicant: Oregon Health & Science University, Portland, OR (US)

(72) Inventors: Clara MOSQUERA-LOPEZ, Portland, OR (US); Peter G. JACOBS, Portland, OR (US); Leah WILSON, Portland, OR (US); Jessica CASTLE, Portland, OR (US)

(21) Appl. No.: 18/858,236

(22) PCT Filed: Apr. 29, 2023
(86) PCT No.: PCT/US23/66417

§ 371 Date: Oct. 18, 2024

Related U.S. Application Data
(60) Provisional application No. 63/363,939, filed on Apr. 29, 2022.

(51) Int. Cl. G16H 20/17; A61M 5/172; G16H 20/60

(52) U.S. Cl. CPC G16H 20/17; A61M 5/1723; G16H 20/60; A61M 2202/07; A61M 2230/201

(57) ABSTRACT

Disclosed is a meal detection and meal size estimation machine learning technology. In some embodiments, the techniques entail applying to a trained multioutput neural network model a set of input features, the set of input features representing glucoregulatory management data, insulin on board, and time of day, the trained multioutput neural network model representing multiple fully connected layers and an output layer formed from first and second branches, the first branch providing a meal detection output and the second branch providing a carbohydrate estimation output; receiving from the meal detection output a meal detection indication; and receiving from the carbohydrate estimation output a meal size estimation.

### RELATED APPLICATION

This application claims priority benefit of U.S. Provisional Patent Application No. 63/363,939, filed Apr. 29, 2022, which is hereby incorporated by reference in its entirety.

### ACKNOWLEDGEMENT OF GOVERNMENT SUPPORT

This invention was made with government support under R01DK120367 and R01DK122583 awarded by the National Institutes of Health. The government has certain rights in the invention.

### TECHNICAL FIELD

This disclosure relates to use of machine learning for detecting meals and estimating their size.

### BACKGROUND INFORMATION

Closed-loop systems for automated insulin delivery (AID) facilitate diabetes management with reduced burden for users. Currently available systems, however, are hybrid in nature and still require the user to count carbohydrates and manually announce meals to the system. Ideally, meal insulin is delivered before meal intake to prevent large postprandial glucose excursions.

Carbohydrate counting is challenging and inaccurate. Current carbohydrate counting methods require a level of numeracy and literacy that might be a barrier for some people with diabetes. In fact, previous research has shown that 49% of meals with less than 30 grams (g) of carbohydrates are overestimated by an average of 25.7±17.2 g, while the majority (64%) of large carbohydrate meals (i.e., those with carbohydrate greater than or equal to 60 g) are underestimated by an average of 53.6±33.8 g. Inaccurate carbohydrate counts that are used for calculation of meal insulin are associated with high prevalence of postprandial hyperglycemia and hypoglycemia leading to suboptimal postprandial glycemic control even with hybrid closed-loop insulin delivery systems.

### SUMMARY OF THE DISCLOSURE

Several attempts at automated meal detection have been described in the literature, including those employing continuous glucose measurements (CGM) and insulin delivery data, and, in some cases, physical activity data. Some of the approaches for automated meal detection described in previously published works include fuzzy logic, Kalman filtering, quantification of the difference between predicted glucose using an autoregressive or other models versus measured CGM values, glucose increase detection, and more recently machine-learning-based detectors using random forest and boosted trees. Sensitivity varies greatly depending on the datasets used and the study protocols, reaching values greater than 90% in some cases. In general, the sensitivity and specificity of many of previously published algorithms have been validated in silico and lack validation of algorithms on real-world meal scenarios or on large real-world free-living datasets.

This disclosure describes machine-learning-based meal detection that may be implemented in a smart device (e.g., smartphones and tablets), in distributed systems including cloud-based computing components, in a closed-loop insulin delivery system, and in combinations thereof.

In some embodiments, a multioutput neural network detects meals and estimates carbohydrate content using CGM and insulin data from people with type 1 diabetes (TID). Input features are derived from a two-hour history of CGM, insulin on board 60 minutes prior to prediction time, and time of day.

This disclosure presents results of the neural network both in silico and on a real-world dataset. Some embodiments detect meals less than 60 minutes after intake. Meals can be detected, and their size can be approximated for use in AID applications for improving time in range with a low false positive rate (e.g., less than one false positive per day). Estimation of meal size in terms of carbohydrate content provides for more accurate calculation of meal insulin doses for individuals with diabetes. Thus, some embodiments include a closed-loop automated hormone delivery system responsive to meal detection and meal size estimation.

Because the embodiments are flexible, without retraining the neural network, the disclosed techniques may be used to detect meals using only CGM for people with and without diabetes. Thus, some embodiments provide robust results in meal detection in people irrespective of whether they have diabetes. In these embodiments, the disclosed techniques may be used in diabetes care and also in the field of nutrition and diet coaching applications.

Automated meal detection from CGM and insulin data provides for: full automation of closed-loop insulin delivery systems, an objective measure of compliance to meal-related study protocols, and reduced user burden when meal reporting is required as part of clinical studies or medical treatments. Additional aspects and advantages will be apparent from the following detailed description of embodiments, which proceeds with reference to the accompanying drawings.

### BRIEF DESCRIPTION OF THE DRAWINGS

To easily identify the discussion of any particular element or act, the most significant digit or digits in a reference number refer to the figure number in which that element is first introduced.

FIG. 1 is a block diagram of an architecture of a multioutput neural network designed for meal detection and meal size estimation.

FIG. 2 is a graph showing the performance of the multioutput neural network of FIG. 1 on simulated data as measured by a receiver operating characteristic (ROC) curve when tested on 40% held-out set of virtual subjects simulated using the Oregon Health & Science University (OHSU) simulator, high-carb meal scenarios.

FIG. 3 is a graph showing the performance of the multioutput neural network on simulated data as measured by an ROC curve when tested on a 40% held-out set of virtual subjects simulated with UVA-Padova and OHSU simulators, low-carb meal scenarios.

FIG. 4 is an example graph of meal detection performance on a Tidepool subject on closed-loop therapy in which the red horizontal line represents P_{TH} equal to 0.86 and the hypoglycemic range (glucose less than 70 mg/dL) is highlighted in red.

FIG. 5 is a confusion matrix chart showing the performance of the multioutput neural network on estimating meal size on simulated data, tested on 40% held-out subjects simulated using the OHSU simulator, high-carb meal scenarios.

FIG. 6 is a confusion matrix chart showing the performance of the multioutput neural network on estimating meal size on simulated data, tested on a 40% held-out set of simulated virtual subjects with UVA-Padova and OHSU simulators, low-carb meal scenarios.

FIG. 7 is a block diagram of a system for detecting meals and estimating their size, according to one embodiment.

FIG. 8 is a pictorial view showing components of the system of FIG. 7 deployed on a person, the components including a smartphone in wireless communication with an insulin pump and a CGM.

FIG. 9 is a flowchart of a method in accordance with one embodiment.

FIG. 10 is a block diagram of a computing device, according to one embodiment.

### DETAILED DESCRIPTION OF EMBODIMENTS

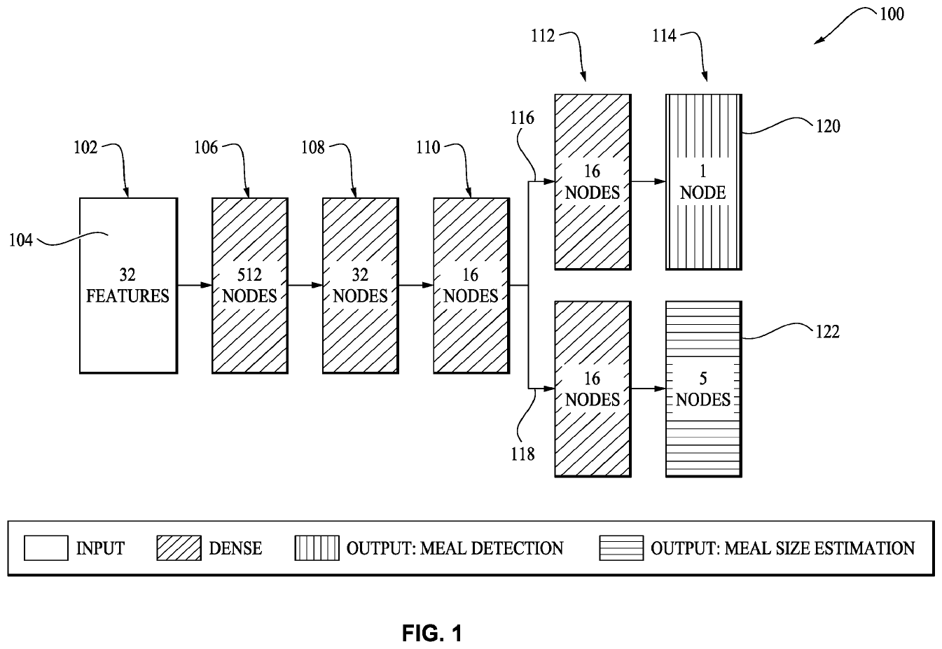

FIG. 1 shows an example architecture of a multioutput neural network 100 designed for meal detection and meal size estimation, according to one embodiment. Multioutput neural network 100 includes two outputs and fully connected neural network (FCNN) layers. FCNN is a series of fully connected layers in which every neuron (also called nodes) in one layer is connected to every neuron in its adjacent layers. For conciseness, however, skilled persons will appreciate that FIG. 1 does not show individual neurons but instead shows layers representing the neurons and their connections with neurons of adjacent layers. With reference to the particular embodiment of multioutput neural network 100, there are six layers described as follows.

A first layer 102 includes 32 (2⁵) neurons corresponding to 32 input features 104 derived from two-hour history of CGM measurements obtained prior to and at prediction time CGMₖ₋ₕ, insulin on board (IOB) one hour before prediction time, and prediction time (i.e., hour of day). The number of features need not be derived using a power of two number—the number of features is designed to capture glucose rises corresponding to intake of meals. Additional features are incorporated to help reduce false positives when calibration occurred.

A descriptive list of input features 104 used for meal detection and size estimation is presented in Table 1. Each of input features 104 described in Table 1 is linearly scaled to fall within a range from (and including) zero to one. Scaling constants were calculated using the training datasets and then used for scaling the validation datasets.

TABLE 1
Features used for meal detection and meal size estimation.

| | | |
|---|---|---|
| | No. of | |
| Description | Features | Specification/Calculation |

Glucoregulatory Management Features

| | | |
|---|---|---|
| glucose at the time of | 1 | f₁ = CGMₖ |
| prediction | | |
| time series of glucose | 12 | f₂₋₁₃ = CGMₖ₋ₕ |
| measurements corresponding | | h ∈ {1, 2, 3, . . . , 12} |
| to one-hour worth of data | | |
| prior to the prediction time | | |
| glucose rate of change (GROC) at the time of | 1 | ${f}_{14} = \frac{{CGM}_{k} - {CGM}_{k - 1}}{Δ t}$ |
| prediction, computed using | | |
| second-order central | | |
| differences in the interior | | |
| points and first-order | | |
| forward or backwards | | |
| differences at the boundaries | | |
| average GROC during the hour prior to prediction | 1 | ${f}_{15} = \frac{1}{13} {\sum }_{h = 0}^{12} {GROC}_{k - h}$ |
| count of GROC values over the hour prior to prediction that are greater than pre- | 8 | ${f}_{16 - 23} = {\sum }_{h = 0}^{12} f ( {GROC}_{k - h} )$ |
| defined thresholds | | $f ( {GROC}_{k - h} ) = \{ \begin{matrix}1 , & if {GROC}_{k} > th \\ 0 , & otherwise\end{matrix}$ |
| | | th ∈ {0.0, 0.5, 1.0, 1.5, 2.0, 2.5, 3.0, 6.0} |
| average glucose over one hour prior to prediction time | 1 | ${f}_{24} = \frac{1}{13} {\sum }_{h = 0}^{12} {CGM}_{k - h}$ |
| average glucose calculated from two hours to one hour prior to prediction time | 1 | ${f}_{25} = \frac{1}{12} {\sum }_{h = 13}^{24} {CGM}_{k - h}$ |
| difference in average glucose during the last half hour vs. the preceding half hour | 1 | ${f}_{26} = \frac{1}{6} {\sum }_{h = 7}^{12} {CGM}_{k - h} - \frac{1}{7} {\sum }_{h = 0}^{6} {CGM}_{k - h}$ |
| average difference between glucose over 30 minutes prior to prediction with | 1 | ${f}_{27} = \frac{1}{6} {\sum }_{h = 0}^{5} ( {CGM}_{k - h} - {CGM}_{k - 6} )$ |
| respect to the glucose value | | |
| exactly 30 minutes prior to | | |
| prediction time | | |
| binary value, set to 1 if the GROC at prediction time is | 1 | ${f}_{28} = \{ \begin{matrix}1 , & if {GROC}_{k} > 5 \\ 0 , & otherwise\end{matrix}$ |
| greater than 5 mg/dL/min | | |
| binary value, set to 1 if the GROC at prediction time is | 1 | ${f}_{29} = \{ \begin{matrix}1 , & if {GROC}_{k} > 7 \\ 0 , & otherwise\end{matrix}$ |
| greater than 7 mg/dL/min | | |

Insulin Features

| | | |
|---|---|---|
| IOB calculated 60 minutes | 1 | f₃₀ = IOBₖ₋₁₂ |
| prior to prediction time | | |

Time Features

| | | |
|---|---|---|
| time of day (hour, 0-23) computed on a unit circle for | 2 | ${f}_{31} = cos ( 2 π \frac{hour}{24} )$ |
| modeling time as a continuous function | | ${f}_{32} = sin ( 2 π \frac{hour}{24} )$ |

IOB was calculated as the weighted sum of prior insulin boluses (B) over the past nine hours using Equation 1, below. In the model described by Equation 1, IOB rises linearly until it reaches a peak at 30 minutes after injection, then it stays constant during one hour before it starts to exponentially decay with a decay constant, ZIOB (for the example results presented here, ZIOB was set equal to 0.012). The IOB formula below has been used in prior publications including the following: Jacobs P G, El Youssef J, Castle J, et al. Automated control of an adaptive bihormonal, dual-sensor artificial pancreas and evaluation during inpatient studies. IEEE Trans Biomed Eng. 2014; 61(10):2569-2581; Jacobs P G, Resalat N, El Youssef J, et al. Incorporating an Exercise Detection, Grading, and Hormone Dosing Algorithm Into the Artificial Pancreas Using Accelerometry and Heart Rate. J Diabetes Sci Technol. 2015; 9(6):1175-1184; and Mosquera-Lopez C, Jacobs PG. Incorporating Glucose Variability into Glucose Forecasting Accuracy Assessment Using the New Glucose Variability Impact Index and the Prediction Consistency Index: An LSTM Case Example. J Diabetes Sci Technol. 2021:19322968211042621.

$${IOB}_{k} = {\sum }_{j = 0}^{6} \frac{j}{6} {B}_{k - 5 j} + {\sum }_{j = 7}^{18} {B}_{k - 5 j} + {\sum }_{j = 19}^{108} {B}_{k - 5 j} {e}^{- ( 5 j ) {Z}_{IOB}} \qquad Equation 1$$

A second layer 106 includes 512 neurons, which are fully connected to the 32 neurons in first layer 102. The 512 neurons are also connected to 32 neurons in a third layer 108. A third layer 108 is fully connected to a fourth layer 110 having 16 nodes. The 16 neurons of fourth layer 110 are fully connected to 32 neurons in a fifth layer 112. A fifth layer 112 is then partly connected to a sixth layer 114 because fifth layer 112 includes a first branch 116 and a second branch 118, each having 16 neurons. Thus, the 16 neurons of first branch 116 are connected to a meal detection output neuron 120 acting as a binary classification for meal detection less than 60 minutes after intake. The 16 neurons of second branch 118 are connected to carbohydrate estimation output neurons 122 acting as a multiclass classification for meal sizes, e.g., categorized into five groups as follows: [0,20) g; [20,40) g; [40,60) g; [60,80) g; and greater than or equal to 80 g.

The architecture of multioutput neural network 100 was determined using a grid search, searching for N layers and hidden nodes for the layers. As noted above for the number of input features, neurons in hidden layers need not be derived using a power of two number.

Multioutput neural network 100 was trained using Python 3.7 and Keras 2.4.0 with Tensorflow backend. L1 regularization with penalty constant of 1e−6 was used in all hidden layers. All weights were randomly initialized using Xavier uniform initializer, and bias were initially set to zero. Adam optimizer with constant learning of 1e−4 and recommended values for the rest of parameters was used to minimize binary and categorical cross-entropy losses for detection and classification outputs, respectively. Training was done with mini batches of size 128. Detection loss and classification loss were equally weighted. For carbohydrate content estimation, the samples in the training dataset were weighted to account for imbalance in the dataset and for penalizing overestimation. Early stopping was implemented to help prevent overfitting.

The datasets included in silico datasets (i.e., low-carbohydrate diet dataset and a high-carbohydrate diet dataset) and a real-world dataset. Each of these is described as follows.

For the two in silico datasets, data were obtained from simulations performed using the following two validated TID simulators: the UVA-Padova simulator, which is approved by the FDA for in silico pre-clinical validation; and a published open-source simulator developed by the present inventors at OHSU. The simulations included 199 virtual subjects for 14 days using real-world meal scenarios from previous studies. Glucose control was simulated using the OHSU's model predictive control (MPC) closed-loop algorithm. Thirty three percent of the meals were simulated without a corresponding insulin bolus. CGM measurements were simulated at a five-minute sampling period. A subject-level split was then included to leave out 40% of the subjects for validation purposes.

The low-carbohydrate diet dataset included 100 UVA-Padova simulator virtual subjects plus 99 OHSU simulator virtual subjects simulated following a real-world low-carbohydrate diet with meals containing, on average, 46.62±27.07 g of carbohydrates. The average carbohydrate content in the validation set was 46.54±26.96 g. These scenarios correspond to the carbohydrate consumption for an individual with diabetes, as some adults with diabetes aim for less than 45-60 g of carbohydrates per meal to maintain postprandial glucose control.

The high-carbohydrate diet dataset included 99 virtual subjects from the OHSU simulator given three meals a day matching the meals simulated by Meneghetti et al. (“Model-Based Detection and Classification of Insulin Pump Faults and Missed Meal Announcements in Artificial Pancreas Systems for Type 1 Diabetes Therapy.” IEEE Trans Biomed Eng. 2021; vol. 68, issue 1; pp. 170-180). To generate this dataset, mealtimes were randomly drawn from uniform distributions in the time intervals 7:00-8:00 for breakfast, 11:30-13:00 for lunch, and 18:30-20:00 for dinner. Similarly, the carbohydrate content of the meals was randomly sampled from the following normal distributions: 58.2±22.5 g for breakfast, 77.7±27.0 g for lunch, and 83.9±32.3 g for dinner. Overall, simulated meals on this high-carbohydrate diet contained 72.46±29.27 g. The average carbohydrate content in the validation set was 72.33±30.10 g.

For the real-world dataset, data from 150 closed-loop users (age 29±16 years; 66 females, 47 males, 37 records with unknown biological sex; 15±12 years since TID diagnosis) from the Tidepool Big Data Donation Program (Tidepool.org, Palo Alto, CA) were used for external real-world validation of the meal detection algorithm. This is a large free-living dataset with time-matched glucose, insulin, and self-reported carbohydrates intake. Data were gathered from multivendor CGM and insulin pump devices through the Tidepool.org platform. Tidepool does not provide information on the make and model of sensors or pumps used during data collection. Like the simulated data, CGM data were collected at a five-minute sampling period. There were 115,106 meals in the dataset indicated by meal bolus insulin entries in their insulin pump logs. While the analysis presumes that the meal insulin was dosed at the time of the meal, many examples were found in which insulin was not dosed for a meal or it was dosed before or after the meal.

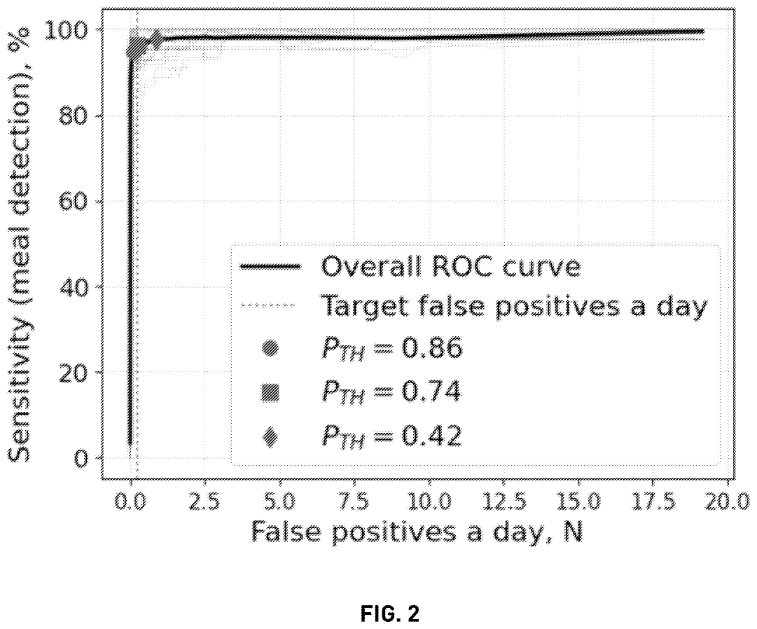

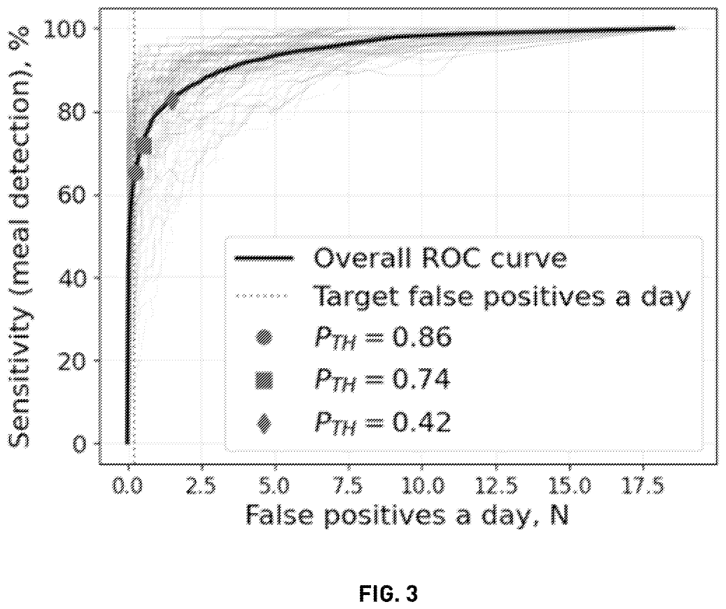

FIG. 2 and FIG. 3 show graphs of the overall performance of meal detection output neuron 120 on the in silico validation dataset using the ROC curve. The validation dataset combined the 40% OHSU held-out participants not used in the algorithm training plus the UVA Padova simulator study participants. FIG. 2 shows the performance on high carbohydrate meals (72.33±30.10 g), and FIG. 3 shows the performance on lower carbohydrate meals (46.54±26.96 g). Individual ROC curves for the OHSU simulator are shown in green and for the UVA-Padova simulator are shown in orange.

Sensitivity and specificity (specificity was measured by the amount of false positive detections per day) were assessed at three selected operating points on the curve. Three operating points for different sensitivity levels are shown in the graphs for different false positives per day. For a target number 0.25 false positives a day, the probability threshold for detecting a meal is P_{TH} equal to 0.86. Since one false positive every four days is considered acceptable clinically, this threshold was used for subsequent analyses.

Based on results in FIG. 2, for higher-carbohydrate meals (i.e., meals with an average carbohydrate content on the higher end of the typical range of 40-65 g per meal consumed by an adult with diabetes), meal detection output neuron 120 (FIG. 1) detects 94.70% of these meals with an average of just 0.11 false positives a day.

FIG. 3 shows that for lower-carbohydrate meals (i.e., meals with an average carbohydrate content on the lower end of the typical range of meals consumed by an adult with diabetes), meal detection output neuron 120 detects 65.44% of these meals with an average of 0.26 false positives a day.

Additional performance metrics considered to assess accurate detection of meals are the detection time calculated as the time difference between the time of meal detection and the actual mealtime, as well as the average carbohydrate content of the meals that were not detected by the algorithm. Table 2 presents the average meal detection time and the average carbohydrate content of meals not detected by the algorithm.

TABLE 2
Average detection time of detected meals and
carbohydrate content of not detected meals.

| | | |
|---|---|---|
| | | Average carbohydrate |
| Meal detection threshold | Detection time | content of not detected meals |
| P_{TH} | Min | g |

Higher-carbohydrate meals

| | | |
|---|---|---|
| 0.86 | 22.93 | 34.38 |
| 0.74 | 22.07 | 29.74 |
| 0.42 | 20.86 | 38.28 |

Lower-carbohydrate meals

| | | |
|---|---|---|
| 0.86 | 27.54 | 27.32 |
| 0.74 | 26.69 | 26.56 |
| 0.42 | 25.38 | 25.18 |

The meal detection algorithm performs better at detecting higher-carbohydrate meals and detects meals about 25 minutes after meal intake. Average detection time for P_{TH} equal to 0.86 was lower for meals with higher-carbohydrate content at 22.93 minutes compared with detection time of meals with lower-carbohydrate content at 27.54 minutes. Meals that were not detected by the algorithm were generally those with low-carbohydrate content (average of 34.38 g for the dataset with higher-carbohydrate contents and 27.32 g for the dataset with meals with lower carbohydrate content).

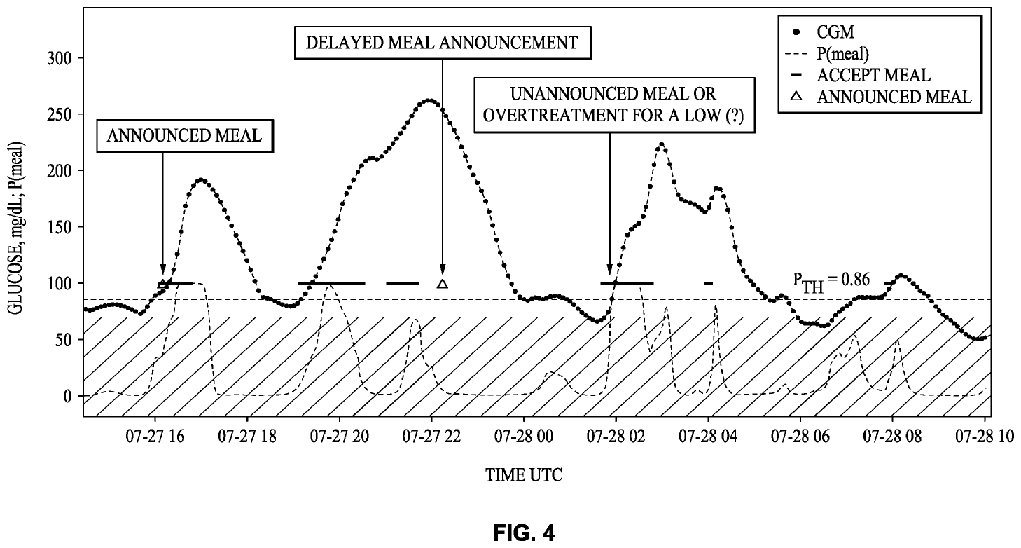

The performance of meal detection output neuron 120 in detecting meals on a real-world glucoregulatory management dataset was also tested. For this dataset, it was found that logged carbohydrates were oftentimes not matched to insulin boluses. Accuracy results for the real-world dataset provided by Tidepool.org are summarized in Table 3 and FIG. 4.

TABLE 3
Meal detection accuracy results on real-
world dataset from Tidepool.org.

| | Approximate sensitivity | | False |
|---|---|---|---|
| Meal detection | % (TP/(TP + FN), where | Confirmed | positives |
| threshold | TP = True positives, | meals | a day |
| P_{TH} | FN = False negatives) | % | N |
| 0.86 | 45.68 | 88.21 | 0.37 |
| 0.74 | 50.35 | 86.12 | 0.51 |
| 0.42 | 59.06 | 80.83 | 0.91 |

In Table 3, the approximate sensitivity metric is the sensitivity, TP/(TP+FN), for those meals which are reported by the Tidepool participants. For different classification thresholds, on the Tidepool dataset, meal detection output neuron 120 achieved 45.68%-59.06% approximate sensitivity. Note that this is an estimate of approximate sensitivity for this dataset since there are many events present in the dataset whereby the person either did not report a meal event or logged it after the meal was consumed. The ground truth has many unannounced meals and meals that were not accurately reported (timing and amount) by the Tidepool participants. Thus, the low approximate sensitivity is due to the ground truth in this dataset being largely unreliable, so it is not known when all meals were consumed. This is clearly demonstrated in FIG. 4 for one Tidepool participant who had one accurate meal announcement, one significantly delayed meal announcement, and one significant rise in glucose that was likely a meal but was not recorded as a meal. For this reason, it is understandable why there is a lower sensitivity of detecting meal events in the real-world Tidepool data.

To better understand if the meals detected by meal detection output neuron 120 were reasonable predictions, the “confirmed meals” metric acts as a heuristic to identify if detected meals exhibit a glucose rise as could be expected following a meal. This heuristic for confirming meals has been used by other groups to identify events that are reasonable estimations of meals. The confirmed meals metric defines those meals detected as having the property that (1) CGM continued to rise after detection and (2) glucose increased more than 20 mg/dL from a 30-minute baseline. Of the meals that were detected by the algorithm, Table 3 shows that 80%-86% of them led to glucose outcomes that indicated a meal had occurred according to the confirmed meal criteria (i.e., a substantial glucose rise from baseline). This implies that while the algorithm could accurately detect about 50%-60% of the Tidepool meal events, for those that it detected, there is a high confidence that these were really meals.

Finally, Table 3 shows that false positives ranged from 0.37-0.91 per day, depending on the choice of the threshold.

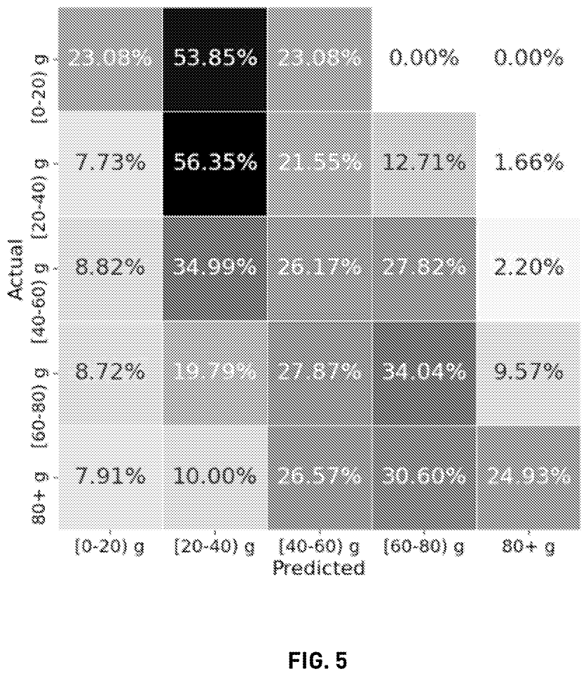

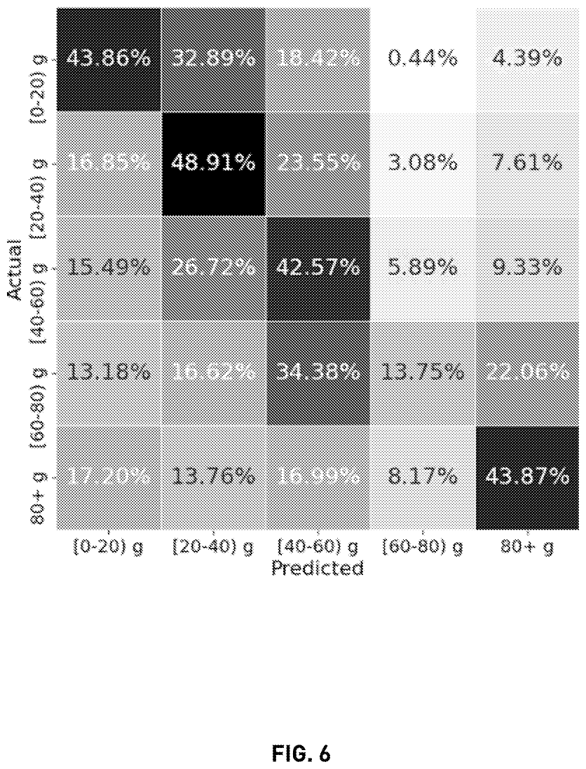

The evaluation of carbohydrate estimation output neurons 122 in estimating meal size is shown in FIG. 5 and FIG. 6 in the form of confusion matrices. A confusion matrix summarizes the percent correct and incorrect predictions made by the algorithm broken down by group. It gives insight not only into the errors being made by the classifier but more importantly the types of errors that are being made (e.g., underestimation and overestimation).

FIG. 5 and FIG. 6 show confusion matrices for accurately detected in silico meals (i.e., true positive detections) at a detection threshold P_{TH} equal to 0.86. It can be observed that most errors are in close categories, and just a small percent of the errors correspond to overestimated carbohydrate content.

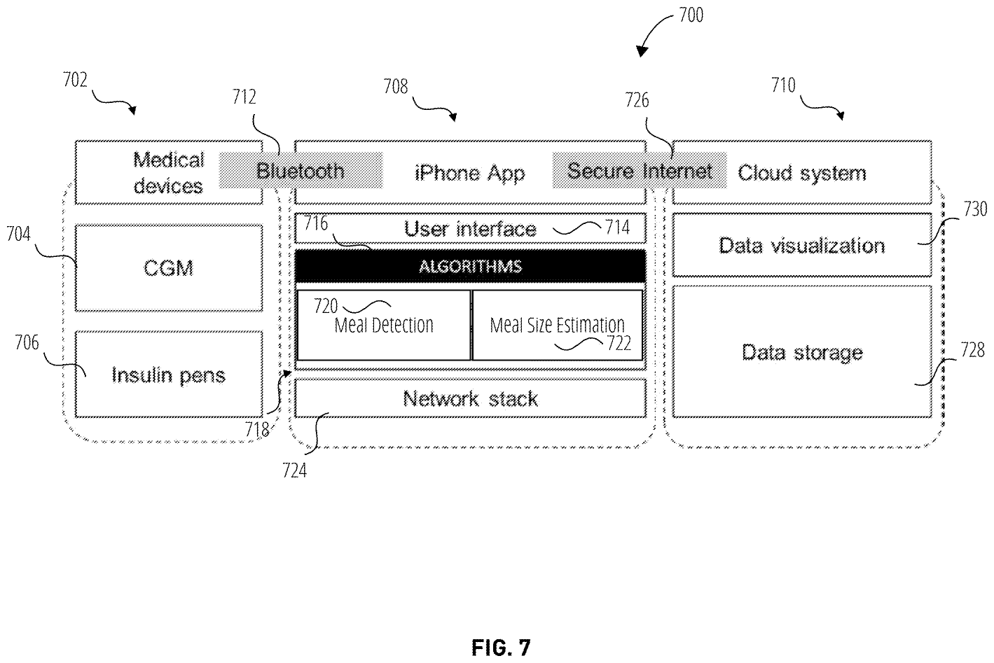

FIG. 7 shows a system 700 for meal detection and size estimation. System 700 includes a wearable glucose sensing and regulating medical device 702 (e.g., CGM 704 or insulin pen 706), mobile device software decision support application 708 (e.g., an iPhone app, smartwatch app, or other smart device app) running on associated user equipment, and a cloud-based software application 710 running on associated computing devices.

Wearable glucose sensing and regulating medical device 702 communicates user data over a wireless personal area network (PAN) connection 712 (e.g., Bluetooth) with mobile device software decision support application 708. For instance, CGM 704 reports some or all of glucoregulatory management data shown in Table 1. In other embodiments, CGM 704 reports raw data from which mobile device software decision support application 708 or cloud-based software application 710 derives glucoregulatory management data shown in Table 1. Similarly, wearable glucose sensing and regulating medical device 702 may report IOB based on tracking use of insulin pen 706 or an insulin pump (or other source of exogenous insulin). The measured or sampled data from wearable glucose sensing and regulating medical device 702, or data derived therefore, is generally referred to as glucose data.

Mobile device software decision support application 708 wirelessly receives the glucose data and renders a user interface 714 that presents to a user glucoregulatory information represented in FIG. 7 in connection with a set of algorithms 716. As described previously, algorithms 716 include a trained multioutput neural network model 718 that provides meal detection 720 and meal size estimation 722, described previously.

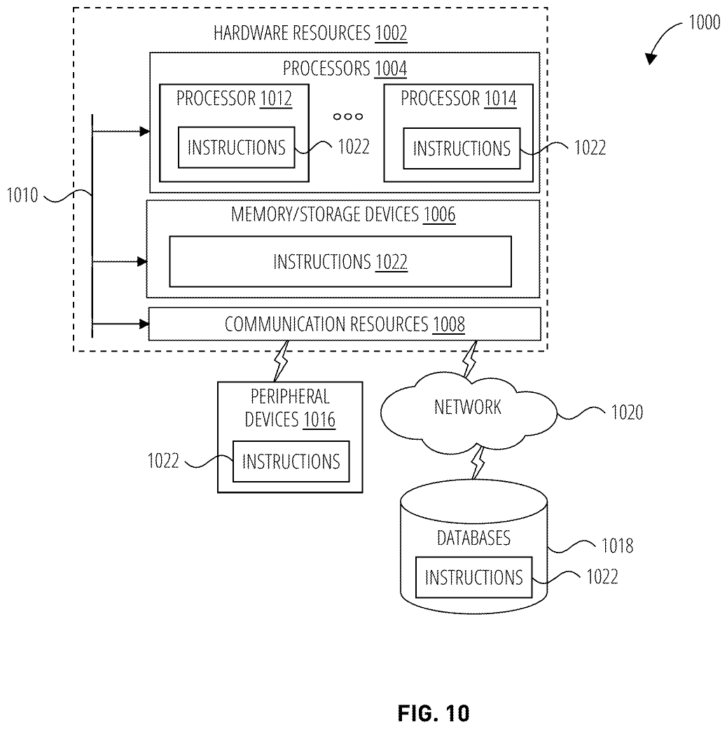

Skilled persons will appreciate that trained multioutput neural network model 718 represents (i.e., models) the structure of multioutput neural network 100 after it has been trained such that each neuron is assigned appropriate weights. The term model in trained multioutput neural network model 718, therefore, would be understood to refer to multioutput neural network 100 that is trained and ultimately compiled in the form of a library or other instructions saved to a computer-readable medium and available for execution on a processor (see, e.g., FIG. 10). An example utility for modeling a trained neural network in source code is described in “Keras2c: A library for converting Keras neural networks to real-time compatible C,” by Conlin et al. Other utilities may also be used for similar purposes. Accordingly, a trained neural network model may include any type of computer instruction or computer-executable code located within a memory device and configured to perform the function of the corresponding trained neural network. In this context, a model may, for instance, include one or more physical or logical blocks of computer instructions, which may be organized as a routine, program, object, component, data structure, etc., that perform the function of the corresponding trained neural network. Skilled persons will also appreciate that the model may be implemented in hardware or firmware instead of or in addition to a software application.

For completeness, mobile device software decision support application 708 also shows lower-layer OS components such as a network stack 724 for communication with cloud-based software application 710. For example, cloud-based software application 710 is configured to receive data from mobile device software decision support applications 708 through a secure internet connection 726. The data may then be stored in data storage 728 used to generate a data visualization 730 for display on user interface 714. In some embodiments, algorithms 716 are implemented in cloud-based software application 710, in which case mobile device software decision support application 708 simply provides data (i.e., input features 104) to cloud-based software application 710. In certain embodiments, trained multioutput neural network model 718 may include disparate instructions stored in different locations of a memory device, different memory devices, or different computers, which together implement the described functionality of the model. Indeed, a model may include a single instruction or many instructions, and may be distributed over several different code segments, among different programs, and across several memory devices. Some embodiments may be practiced in a distributed computing environment where tasks are performed by a remote processing device linked through a communications network. In a distributed computing environment, models may be located in local or remote memory storage devices. In addition, data being tied or rendered together in a database record may be resident in the same memory device, or across several memory devices, and may be linked together in fields of a record in a database across a network.

Once mobile device software decision support application 708 or cloud-based software application 710 detects a meal or estimate its size, alerts may be provided to the user via user interface 714. In fully closed-loop AID systems, wearable glucose sensing and regulating medical device 702 may be triggered to provide insulin via insulin pen 706. For instance, with reference to Table 4 below, the performance of meal detection 720 and meal size estimation 722 algorithms 716 was evaluated on the OHSU in silico simulator within the context of a fully automated AID system. Data were used from 20 real-world meal scenarios acquired over a four-day period from an out-patient closed-loop study. For certain scenarios, insulin was not dosed for any of these meals such that meal detection output neuron 120 was used instead to dose insulin automatically for these meals. The amount of insulin dosed in response to meal detection output neuron 120 was based on carbohydrate estimation output neurons 122 used to estimate the carbohydrate content of each detected meal to calculate the amount of insulin for the meal. For instance, the algorithm was designed to be used within a system whereby the user is first notified of the meal detection and the estimated meal carbohydrate content. Once the user confirms the detection and carbohydrate amount, the meal insulin is delivered. If the user confirms immediately, 75% (or other fraction) of the insulin is dosed. If the user confirms up to 20 minutes after the algorithm notifies the user, the amount of insulin dosed is reduced linearly by 1% per minute down to 55% of the meal insulin if the user confirms 20 minutes after detection. The following five different scenarios were evaluated to quantify the performance of the algorithm under different conditions.

Scenario 1: Participant dosed correct amount of insulin for each meal. This is the case of hybrid closed-loop system whereby meal insulin is always correctly dosed at the time that the meal is taken. The meal detection algorithm is not used in this scenario.

Scenario 2: Participants did not dose for meals and no automated meal detection/dosing algorithm is used. This is a fully automated closed-loop mode whereby no meal detection is done, and the system relies on the control algorithm's basal insulin delivery for glucose control. This is a closed-loop algorithm for glucose control, whereby a control algorithm doses insulin based on CGM readings.

Scenario 3: Participants did not dose for meals, perfect meal detection and meal amount estimation at 25 minutes post-meal was simulated, and participant confirms the dose immediately. This is the case where meal detection is simulated with a fixed detection time of 25 minutes after meals and the user responds immediately to the missed meal notification and doses 75% of the correct amount of insulin based on accurate carbohydrate counting.

Scenario 4: Participants did not dose for meals, perfect detection and meal size estimation at 25-minutes post meal was simulated, and participant confirmed the dose 20 minutes after detection. This is the case in which meal detection time is fixed at 25 minutes after meal intake and the user responds to the notification of an announced meal 20 minutes after the alarm (i.e., 45 minutes after the meal) and doses 55% of the correct amount of insulin.

Scenario 5: Participants did not dose for meals, the proposed meal detection algorithm detected the meal and estimated the meal size, and the participants confirmed the meal insulin dose immediately. This is the fully AID mode whereby the meal detection algorithm is used to automatically dose 75% of meal insulin based on the carbohydrate content of detected meals estimated by the proposed algorithm.

Table 4 shows the following outcome measures for the population of 99 OHSU in silico test participants: percent time in hypoglycemia (less than 70 mg/dL), number of rescue carbohydrates per day, percent time in target range (70-180 mg/dL), mean glucose, high blood glucose index (HBGI), low blood glucose index (LBGI), percent time in extreme hypoglycemia (less than 54 mg/dL), and total daily insulin requirement (TDIR).

In terms of results, Table 4 shows an example in which meal detection 720 and meal size estimation 722 algorithms 716 compare in terms of glucose control metrics, with perfect detection and carbohydrate counting scenarios and hybrid closed-loop glucose control. When algorithms 716 are used in a fully automated closed-loop in silico trial, automated meal detection improved time in target range from 71.17% (Scenario 2, or no dosing for meals) to 77.10% (Scenario 5, automated meal detection and carbohydrate estimation), maintaining percent time in hypoglycemia of less than 2%.

TABLE 4
Comparative analysis of simulation scenarios with and without
automated meal detection and meal size estimation.

| Outcome metrics | Simulation scenarios | | | | |
|---|---|---|---|---|---|
| (AVERAGE ± STD) | Scenario 1 | Scenario 2 | Scenario 3 | Scenario 4 | Scenario 5 |
| Time in | 0.16 ± 0.48 | 0.01 ± 0.09 | 0.04 ± 0.13 | 0.01 ± 0.04 | 0.37 ± 0.55 |
| hypoglycemia, % | | | | | |
| Rescue carbs per | 0.14 ± 0.38 | 0.02 ± 0.09 | 0.03 ± 0.11 | 0.01 ± 0.04 | 0.28 ± 0.35 |
| day, N | | | | | |
| Time in target | 88.84 ± 8.09 | 71.17 ± 12.00 | 78.4 ± 10.10 | 75.19 ± 10.40 | 77.10 ± 9.62 |
| range, % | | | | | |
| Time in | 11.00 ± 8.17 | 28.82 ± 12.00 | 21.5 ± 10.10 | 24.80 ± 10.40 | 22.53 ± 9.72 |
| hyperglycemia, % | | | | | |
| Average glucose, | 144.55 ± 10.92 | 168.72 ± 25.10 | 158.2 ± 14.80 | 163.22 ± 17.50 | 157.17 ± 15.76 |
| mg/dL | | | | | |
| HBGI | 3.44 ± 1.59 | 8.06 ± 4.92 | 5.69 ± 2.71 | 6.66 ± 3.34 | 6.08 ± 2.77 |
| LBGI | 0.82 ± 0.67 | 0.51 ± 0.23 | 0.67 ± 0.45 | 0.56 ± 0.28 | 1.34 ± 0.89 |
| Time in extreme | 0.01 ± 0.07 | 0.00 ± 0.00 | 0.00 ± 0.00 | 0.00 ± 0.00 | 0.04 ± 0.15 |
| hypoglycemia, % | | | | | |
| TDIR, units | 41.47 ± 15.72 | 38.50 ± 15.30 | 39.60 ± 15.30 | 39.00 ± 15.20 | 39.94 ± 15.11 |

Results in Table 4 indicate that if a meal is accurately detected within 25-45 minutes of the meal occurring (Scenarios 3 and 4) and dosed 75%-55% of their insulin depending on the timing of the bolus insulin, time in range can be improved and there is no increased risk of postprandial hypoglycemia, even when dosing 45 minutes after the meal intake.

Percent time in hypoglycemia increased slightly between these scenarios, however, even with automated dosing, percent time in hypoglycemia stayed well below the standard target of 4% time in hypoglycemia. The small increase in hypoglycemia was not unexpected in that the meal detection algorithm can sometimes overestimate the size of the meal, and there is inherently some increased risk of hypoglycemia when delaying meal insulin. The impact of meal size misestimation can be better understood by comparing Scenario 3 (when the exact amount of meal was dosed 25 minutes after the meal) with Scenario 5 (when the meal detector and estimator were used to dose meal insulin). Scenario 5 yielded higher % time in hypoglycemia (0.37%) compared with Scenario 3 (0.04%).

Results in Table 3 indicate that a typical meal could be detected in about 25 minutes. Delayed detection is a consequence of delays in carbohydrate absorption and inherent delay glucose in interstitium relative to blood glucose. In the analyses, on average, a detectable glucose rise due to meal intake occurs after 20 minutes of consuming a meal. Therefore, it is unlikely that insulin could be dosed for a meal using a CGM-based automated system any sooner than 20 minutes after the meal was consumed. Results presented here show that delayed insulin dosing using an automated meal detection algorithm at about 25 minutes post-meal (Scenario 5) but even as long as 45 minutes after the meal (Scenario 4) provides benefit in terms of increased percentage time in range compared with if the person just relies on traditional automated closed-loop insulin dosing (Scenario 2).

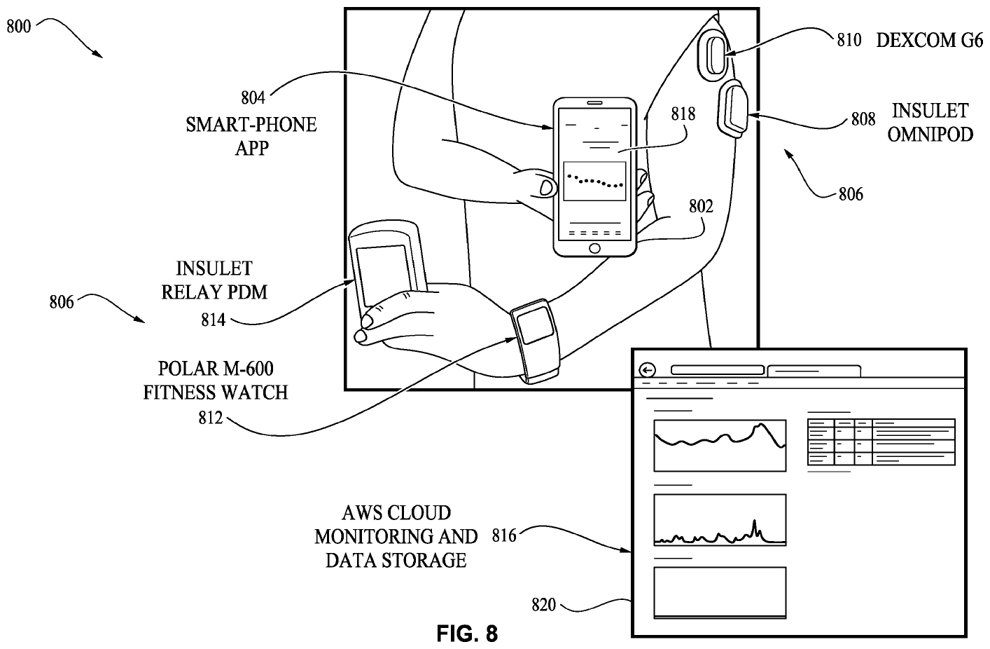

FIG. 8 shows a system 800 including several components akin to those of FIG. 7. For instance, a Samsung smartphone 802 is configured (e.g., using a smart-phone app 804) to perform meal detection 720 and meal size estimation 722. Smartphone 802 is configured to communicate with wearable glucose sensing and regulating medical devices 806, which include an Insulet Omnipod insulin pump 808 (configured to implement features described previously with reference to insulin pen 706) and a Dexcom G6 sensor 810 (or similar device configured to implement features described previously with reference to CGM 704). Also shown is an M-600 fitness watch 812 for quantifying exercise. Wearable glucose sensing and regulating medical devices 806 also include an Insulet relay personal diabetes manager (PDM) 814. Data is stored to an Amazon Web Services (AWS) data repository 816 (e.g., AWS Cloud monitoring and data storage).

In one aspect, system 800 is deployed for meal detection and includes one or more wearable glucose sensing and regulating medical devices 806 coupled to a user and configured to generate glucose data for a set of input features. As explained previously, the set of input features have glucoregulatory, insulin, and associated time of day feature. A smart device (smartphone 802) is configured to wirelessly receive from the one or more wearable glucose sensing and regulating medical devices the glucose data and to provide the set of input features to a trained machine learning model. The trained machine learning model including multiple connected layers and an output layer providing a meal detection output. System also includes a user interface 818 (e.g., on smartphone 802 or other device) configured to present to the user, based on the meal detection output, a meal detection indication.

System 800 may also include a remote server for hosting the trained machine learning model and receiving the set of input features. For instance, a data visualization 820 is presented based on data repository 816. Data visualization 820 may be presented using user interface 818 or another interface in a SaaS platform.

System 800 may also include the trained machine learning model being a trained multioutput machine learning model with the output layer formed from first and second branches. In this example, the first branch provides the meal detection output and the second branch providing a carbohydrate estimation output.

System 800 may also include the multiple connected layers being fully connected layers.

System 800 may also include the trained machine learning model having one or more of a neural network, a random forest model, a support vector regression model, and a logistic regression model.

System 800 may also include the trained machine learning model being trained using one or more of an ordinary differential equation (ODE) in silico model of glucose metabolism and real-world human glucose, insulin, and nutrition data.

System 800 may also include the trained machine learning model being configured to predict categories of meal sizes or an actual meal amount.

System 800 may also include the trained machine learning model providing a probability estimate of a likelihood of a meal having occurred.

System 800 may also include the smart device being configured to receive a series of periodic glucose measurement samples, and derive one or more glucoregulatory features based on the series of glucose measurement samples.

System 800 may also include the smart device being configured to receive insulin bolus data, and calculate one or more insulin features based on a weighted sum of amounts in the insulin bolus data over a predetermined time.

System 800 may also include the user interface being configured to notify the user of a meal detection event, and receive a user confirmation of the meal detection event.

System 800 may also include one or more wearable glucose sensing and regulating medical devices 806 including a continuous glucose monitoring (CGM) device.

System 800 may also include the smart device being configured to wirelessly receive, via a wireless personal area network from one or more wearable glucose sensing and regulating medical devices 806, the glucose data including at least a portion of the set of input features.

System 800 may also include the smart device being configured to initiate delivery of insulin to a user responsive to the meal size estimation.

System 800 may also include one or more wearable glucose sensing and regulating medical devices 702 including an insulin pen or an insulin pump.

System 800 may also include user interface 818 being configured to present to the user, based on the carbohydrate estimation output, a meal size estimation.

System 800 may also include the trained machine learning model providing a probability estimate of the meal size estimation.

System 800 may also include the smart device being configured to determine, based on one or both the meal detection output and the carbohydrate estimation output, an amount of insulin to dose to a person requiring exogenous insulin delivery.

System 800 may also include the meal size estimation being used within decision support application 804 executed by the smart device to estimate whether a meal was consumed prior to meal insulin dosing.

System 800 may also include the smart device being configured to provide the meal size estimation to a weight-loss coaching application.

System 800 may also include the smart device being configured to initiate delivery of a fraction of a requisite amount of meal insulin to the user automatically or in response to reception of the user confirmation.

System 800 may also include the smart device being configured to determine the fraction as a function of time based on a time after the user confirmation.

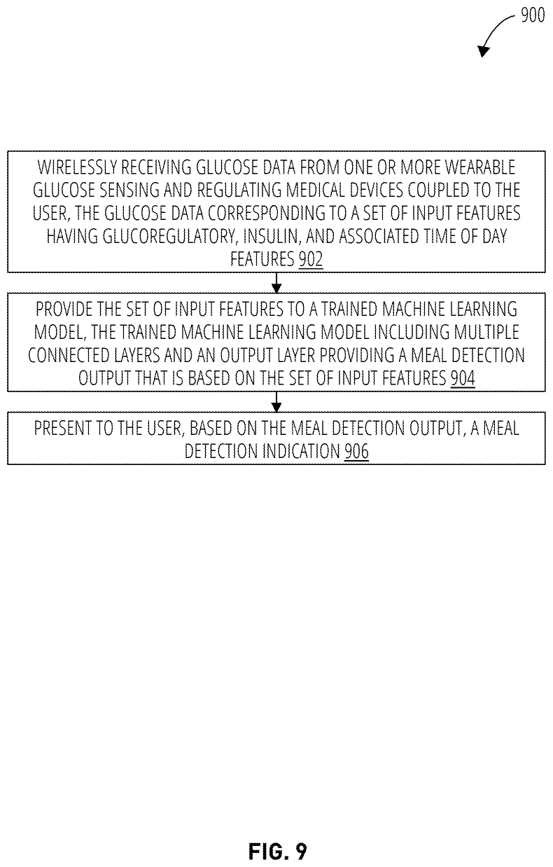

FIG. 9 shows a process 900 for meal detection. In block 902, process 900 entails wirelessly receiving glucose data from one or more wearable glucose sensing and regulating medical devices coupled to the user, the glucose data corresponding to a set of input features having glucoregulatory, insulin, and associated time of day features. In block 904, process 900 provides the set of input features to a trained machine learning model, the trained machine learning model including multiple connected layers and an output layer providing a meal detection output that is based on the set of input features. In block 906, process 900 presents to the user, based on the meal detection output, a meal detection indication.

Process 900 may also include the trained machine learning model being a trained multioutput machine learning model with the output layer formed from first and second branches, the first branch providing the meal detection output and the second branch providing a carbohydrate estimation output.

Process 900 may also include presenting to the user, based on the carbohydrate estimation output, a meal size estimation.

Process 900 may also include the trained machine learning model providing a probability estimate for the meal size estimation.

Process 900 may also include determining, based on one or both the meal detection output and the carbohydrate estimation output, an amount of insulin to dose to a person requiring exogenous insulin delivery.

Process 900 may also include the meal size estimation being used within a decision support application executed by the smart device to estimate whether a meal was consumed prior to meal insulin dosing.

Process 900 may also include providing the meal size estimation to a weight-loss coaching application.

Process 900 may also include the multiple connected layers being fully connected layers.

Process 900 may also include the trained machine learning model having one or more of a neural network, a random forest model, a support vector regression model, and a logistic regression model.

Process 900 may also include the trained machine learning model being trained using one or more of an ordinary differential equation (ODE) in silico model of glucose metabolism and real-world human glucose, insulin, and nutrition data.

Process 900 may also include the trained machine learning model being configured to predict categories of meal sizes or an actual meal amount.

Process 900 may also include the trained machine learning model providing a probability estimate of a likelihood of a meal having occurred.

Process 900 may also include receiving a series of periodic glucose measurement samples, and deriving one or more glucoregulatory features based on the series of glucose measurement samples.

Process 900 may also include receiving insulin bolus data, and calculating one or more insulin features based on a weighted sum of amounts in the insulin bolus data over a predetermined time.

Process 900 may also include notifying the user, via a user interface, of a meal detection event, and receiving a user confirmation of the meal detection event.

Process 900 may also include initiating delivery of a fraction of a requisite amount of meal insulin to the user automatically or in response to reception of the user confirmation.

Process 900 may also include determining the fraction as a function of time based on a time after the user confirmation.

Process 900 may also include the one or more wearable glucose sensing and regulating medical devices having a continuous glucose monitoring (CGM) device.

Process 900 may also include the one or more wearable glucose sensing and regulating medical devices having an insulin pen or an insulin pump.

Process 900 may also include wirelessly receiving the glucose data by receiving at least a portion of the set of input features via a wireless personal area network from the one or more wearable glucose sensing and regulating medical devices.

Process 900 may also include initiating delivery of insulin to a user in response to the meal size estimation.

Process 900 may also include providing the set of input features by transmitting the set of input features to the trained machine learning model hosted by a remote server.

FIG. 10 is a block diagram illustrating components 1000, according to some example embodiments, able to read instructions from a machine-readable or computer-readable medium (e.g., a non-transitory machine-readable storage medium), and perform any one or more of the methods discussed herein (e.g., process 900, FIG. 9). For example, hardware resources 1002 may be embodied in a smartwatch, server, tablet computer, or patient-connected device that provides the ability to measure a physiological signal, provide some analysis of that signal, transmit information about that signal, or support a user interface to provide information about that signal. This includes an equivalent functional combination, for example a watch that can measure a physiological signal and transmit the data to a computer (including a smartphone), where the computer provides analysis and user interface functions.

Specifically, FIG. 10 shows a diagrammatic representation of hardware resources 1002 including one or more processors 1004 (or processor cores), one or more memory/storage devices 1006, and one or more communication resources 1008, each of which may be communicatively coupled via a bus 1010.

Processors 1004 (e.g., a central processing unit (CPU), a reduced instruction set computing (RISC) processor, a complex instruction set computing (CISC) processor, a graphics processing unit (GPU), a digital signal processor (DSP) such as a baseband processor, an application specific integrated circuit (ASIC), another processor, or any suitable combination thereof) may include, for example, a processor 1012 and a processor 1014.

Memory/storage devices 1006 may include main memory, disk storage, or any suitable combination thereof. Memory/storage devices 1006 may include, but are not limited to, any type of volatile or non-volatile memory such as dynamic random access memory (DRAM), static random-access memory (SRAM), erasable programmable read-only memory (EPROM), electrically erasable programmable read-only memory (EEPROM), Flash memory, solid-state storage, etc.

Communication resources 1008 may include interconnection or network interface components or other suitable devices to communicate with one or more peripheral devices 1016 or one or more databases 1018 via a network 1020. For example, communication resources 1008 may include wired communication components (e.g., for coupling via a Universal Serial Bus (USB)), cellular communication components, NFC components, Bluetooth® components (e.g., Bluetooth® Low Energy), Wi-Fi® components, and other communication components.

Instructions 1022 may comprise software, a program, an application, an applet, an app, or other executable code for causing at least any of processors 1004 to perform any one or more of the methods discussed herein (e.g., process 900, FIG. 10). In some embodiments, instructions 1022 include aspects of trained multioutput neural network model 718 (FIG. 7) including one or both meal detection 720 (FIG. 7) and meal size estimation 722 (FIG. 7) stored in memory/storage devices 1006.

Instructions 1022 may reside, completely or partially, within at least one of processors 1004 (e.g., within the processor's cache memory), memory/storage devices 1006, or any suitable combination thereof. Furthermore, any portion of instructions 1022 may be transferred to hardware resources 1002 from any combination of peripheral devices 1016 or databases 1018. Accordingly, the memory of processors 1004, memory/storage devices 1006, peripheral devices 1016, and databases 1018 are examples of computer-readable and machine-readable media.

Skilled persons will appreciate that many changes may be made to the details of the above-described embodiments without departing from the underlying principles of the invention. The scope of the present invention should, therefore, be determined only by claimed inventions and equivalents thereof.

What is claimed is:

1. A method, performed by a smart device of a user, for meal detection, comprising:
wirelessly receiving glucose data from one or more wearable glucose sensing and regulating medical devices coupled to the user, the glucose data corresponding to a set of input features having glucoregulatory, insulin, and associated time of day features;
providing the set of input features to a trained machine learning model, the trained machine learning model including multiple connected layers and an output layer providing a meal detection output that is based on the set of input features; and
presenting to the user, based on the meal detection output, a meal detection indication.

2. The method of claim 1, in which the trained machine learning model is a trained multioutput machine learning model with the output layer formed from first and second branches, the first branch providing the meal detection output and the second branch providing a carbohydrate estimation output.

3. The method of claim 2, further comprising presenting to the user, based on the carbohydrate estimation output, a meal size estimation.

4. The method of claim 3, in which the trained machine learning model provides a probability estimate for the meal size estimation.

5. The method of claim 3, further comprising determining, based on one or both the meal detection output and the carbohydrate estimation output, an amount of insulin to dose to a person requiring exogenous insulin delivery.

6. The method of claim 3, in which the meal size estimation is used within a decision support application executed by the smart device to estimate whether a meal was consumed prior to meal insulin dosing.

7. The method of claim 3, further comprising providing the meal size estimation to a weight-loss coaching application.

8. The method of claim 1, in which the multiple connected layers are fully connected layers.

9. The method of claim 1, in which the trained machine learning model is one or more of a neural network, a random forest model, a support vector regression model, and a logistic regression model.

10. The method of claim 1, in which the trained machine learning model is trained using one or more of an ordinary differential equation (ODE) in silico model of glucose metabolism and real-world human glucose, insulin, and nutrition data.

11. The method of claim 1, in which the trained machine learning model is configured to predict categories of meal sizes or an actual meal amount.

12. The method of claim 1, in which the trained machine learning model provides a probability estimate of a likelihood of a meal having occurred.

13. The method of claim 1, further comprising:
receiving a series of periodic glucose measurement samples; and
deriving one or more glucoregulatory features based on the series of glucose measurement samples.

14. The method of claim 1, further comprising:
receiving insulin bolus data; and
calculating one or more insulin features based on a weighted sum of amounts in the insulin bolus data over a predetermined time.

15. The method of claim 1, in which the presenting comprises:
notifying the user, via a user interface, of a meal detection event; and
receiving a user confirmation of the meal detection event.

16. The method of claim 15, further comprising initiating delivery of a fraction of a requisite amount of meal insulin to the user automatically or in response to reception of the user confirmation.

17. The method of claim 16, further comprising determining the fraction as a function of time based on a time after the user confirmation.

18. The method of claim 1, in which the one or more wearable glucose sensing and regulating medical devices include a continuous glucose monitoring (CGM) device.

19. The method of claim 1, in which the one or more wearable glucose sensing and regulating medical devices include an insulin pen or an insulin pump.

20. The method of claim 1, in which the wirelessly receiving the glucose data comprises receiving at least a portion of the set of input features via a wireless personal area network from the one or more wearable glucose sensing and regulating medical devices.

21. The method of claim 1, further comprising initiating delivery of insulin to a user in response to the meal size estimation.

22. The method of claim 1, in which the providing the set of input features comprises transmitting the set of input features to the trained machine learning model hosted by a remote server.

23. A system for meal detection, comprising:
one or more wearable glucose sensing and regulating medical devices coupled to a user and configured to generate glucose data for a set of input features, the set of input features having glucoregulatory, insulin, and associated time of day features;
a smart device configured to wirelessly receive from the one or more wearable glucose sensing and regulating medical devices the glucose data and to provide the set of input features to a trained machine learning model, the trained machine learning model including multiple connected layers and an output layer providing a meal detection output; and
a user interface configured to present to the user, based on the meal detection output, a meal detection indication.

24. The system of claim 23, further comprising a remote server for hosting the trained machine learning model and receiving the set of input features.

25. The system of claim 23, in which the trained machine learning model is a trained multioutput machine learning model with the output layer formed from first and second branches, the first branch providing the meal detection output and the second branch providing a carbohydrate estimation output.

26. The system of claim 25, in which the user interface is configured to present to the user, based on the carbohydrate estimation output, a meal size estimation.

27. The system of claim 26, in which the trained machine learning model provides a probability estimate of the meal size estimation.

28. The system of claim 26, in which the smart device is configured to determine, based on one or both the meal detection output and the carbohydrate estimation output, an amount of insulin to dose to a person requiring exogenous insulin delivery.

29. The system of claim 26, in which the meal size estimation is used within a decision support application executed by the smart device to estimate whether a meal was consumed prior to meal insulin dosing.

30. The system of claim 26, in which the smart device is configured to provide the meal size estimation to a weight-loss coaching application.

31. The system of claim 23, in which the multiple connected layers are fully connected layers.

32. The system of claim 23, in which the trained machine learning model is one or more of a neural network, a random forest model, a support vector regression model, and a logistic regression model.

33. The system of claim 23, in which the trained machine learning model is trained using one or more of an ordinary differential equation (ODE) in silico model of glucose metabolism and real-world human glucose, insulin, and nutrition data.

34. The system of claim 23, in which the trained machine learning model is configured to predict categories of meal sizes or an actual meal amount.

35. The system of claim 23, in which the trained machine learning model provides a probability estimate of a likelihood of a meal having occurred.

36. The system of claim 23, in which the smart device is configured to:
receive a series of periodic glucose measurement samples; and
derive one or more glucoregulatory features based on the series of glucose measurement samples.

37. The system of claim 23, in which the smart device is configured to:
receive insulin bolus data; and
calculate one or more insulin features based on a weighted sum of amounts in the insulin bolus data over a predetermined time.

38. The system of claim 23, in which the user interface is configured to:
notify the user of a meal detection event; and
receive a user confirmation of the meal detection event.

39. The system of claim 38, in which the smart device is configured to initiate delivery of a fraction of a requisite amount of meal insulin to the user automatically or in response to reception of the user confirmation.

40. The system of claim 39, in which the smart device is configured to determine the fraction as a function of time based on a time after the user confirmation.

41. The system of claim 23, in which the one or more wearable glucose sensing and regulating medical devices include a continuous glucose monitoring (CGM) device.

42. The system of claim 23, in which the one or more wearable glucose sensing and regulating medical devices include an insulin pen or an insulin pump.

43. The system of claim 23, in which the smart device is configured to wirelessly receive, via a wireless personal area network from the one or more wearable glucose sensing and regulating medical devices, the glucose data including at least a portion of the set of input features.

44. The system of claim 23, in which the smart device is configured to initiate delivery of insulin to a user responsive to the meal size estimation.
